(19) United States
(12) Patent Application Publication
(10) Pub. No.: US 2010/0274246 A1
(43) Pub. Date: Oct. 28, 2010

(54) EXPANDABLE INTRAMEDULLARY NAIL FOR SMALL BONE FIXATION

(75) Inventors: Mordechay Beyar, Herzlia Pituach (IL); Oren Globerman, Herzlia Pituach (IL)

Correspondence Address: MARTIN D. MOYNIHAN d/b/a PRTSI, INC., P.O. BOX 16446, ARLINGTON, VA, 22215, US

(73) Assignee: Oren Globerman, Herzlia Pituach (IL)

(21) Appl. No.: 12/676,946

(22) PCT Filed: May 7, 2008
(86) PCT No.: PCT/IL08/00635

§ 371 Date: Jun. 29, 2010

Related U.S. Application Data
(60) Provisional application No. 60/917,125, filed on May 10, 2007.

(51) Int. Cl. A61B 17/58

(52) U.S. Cl. 606/63

(57) ABSTRACT

The present invention provides method and device for fixating a fractured bone by inflating an intramedullary nail using a manually operated nail inflation syringe. Optionally, the nail is provided attached to an insertion device that may also function as the inflation syringe. The two attached items are preferably provided as a single detachable element—sterile and ready for use.

### RELATED APPLICATIONS

The present application claims the benefit under 119(e) of 60/917,125 filed May 10, 2007, the disclosure of which is incorporated herein by reference.

### FIELD OF THE INVENTION

The present invention relates to devices and methods for fixating small bones.

### BACKGROUND OF THE INVENTION

Fractures of small long bones, such as metacarpal, metatarsal bones, clavicle, ulna, radius and fibula, are very common. The size and location of these bones make it difficult to provide traditional fixations healing procedures such as splinting and casting. There is also a rising need to avoid invasive surgical procedures as requested when using fixating bolts, pins with or without combination with plates. A further need is the ability to prevent any relative movements or immobilizations of the bone fractures, including but not limited to lateral movements, rotation and torsion movements and bending movements.

Newer less invasive techniques include boring of the metacarpal bone followed by inserting a pin or a K-wire in order to fasten bone fractures.

U.S. Pat. No. 6,273,892 to Orbay et al., the disclosure of which is fully incorporated herein by reference, describes a nailing device that promotes relatively parallel insertion and fixation technique by providing a drill shaft having a groove for guiding a fixation pin. In order to prevent rotational movements, several fixation pins are intrduced, their proximal ends are bent and a cylindrical collet is positioned over their exposed proximal ends in order to constrain the pins together.

U.S. Pat. No. 6,127,597, the disclosure of which is fully incorporated herein by reference, describes an inflatable nail for fixating fractured long bones, said nail includes longitudinal bars along its axis that are necessary for rotational stability and for 4-point strength.

### SUMMARY OF THE INVENTION

The present invention provides method and device for fixating small bones, as for example in order to treat a broken small bone. In an exemplary embodiment of the invention, said small bones may include but are not limited to a fractured fibula, ulna, radius, clavicle, metacarpal, metatarsal, or phalangeal bones.

An aspect of some embodiments of the invention relates to an expandable fixation nail intended for insertion into a medullar canal of a small bone when in a first collapsed formation and then to be expanded to a preferred expanded formation.

In an exemplary embodiment of the invention, the fixation nail is substantially tubular in collapsed and/or expanded formation. Optionally, the fixation nail is provided as a crimped tubular element. Optionally, the nail has an inner passage (i.e. bore) and can be inflated to at least partial expanded formation. Optionally, said inflation is achieved by providing hydraulic or pneumatic fluid into the nail and through its inner bore, under preferred, optionally predetermined, pressure. Optionally, at least one area or point of the nail goes though plastic deformation when the nail transformed from a first to a second formation. Optionally, said deformation is at least partially elastic.

Optionally, the fixation nail further includes interlocking means along its length and/or on its distal side.

In an exemplary embodiment of the invention, at least one expanded segment of the expanded nail takes the form of the adjacent medullar canal surface as it is pressed against it under sufficient radial forces. Since that the medullar canal is substantially non-uniform along its axis, two distant expanded nail segments will optionally take different size and/or form when nail is expanded and this may contribute to the overall resistance to relative movements of the nail with respect to the treated small bone and/or to bending or torsion stresses in the nail.

In an exemplary embodiment of the invention, the nail includes friction means for preventing slipping of the nail with respect of bone interface. Optionally, said friction means include but are not limited to: roughened textures, spikes and/or fins; said fins may extend longitudinally and/or circumferentially. In a preferred exemplary embodiment of the invention, said friction means are functional when the nail is in at least partial expanded formation. Preferably, said friction means improves rotational and/or longitudinal immobilization stability. In an exemplary embodiment of the invention, when the nail transforms from a first crimped formation to an expanded formation (e.g. by inflation), a residual crimped-like pattern remains; said residual pattern further improves immobilization stability. Optionally, friction means, such as spikes, fins or bars, are attached to the nail outer surface, as for example by welding or soldering.

In an exemplary embodiment of the invention, the fixation nail is provided attached to an insertion device. Optionally, said insertion device includes a shaft having a longitudinal axis and connecting means on its distal end capable of connecting with the fixation nail proximal end. Optionally, said connection with nail is achieved by screwing and/or by gripping, but can be maintained by any other connecting means known to art.

Optionally, said insertion device also serves as a channel for hydraulic or pneumatic fluid. Optionally, the insertion device shaft is a tube-like element having an inner and an outer diameter. Optionally, the insertion device is provided with hydraulic fluid bounded between the nail at the distal side and a sealing element on the proximal side. Optionally, said sealing element is a slidable piston. Optionally, said piston is a floating piston having a rigid tubular core coupled with an O-ring sealing. Optionally, the piston has a relatively constant outer diameter which is substantially equivalent to the inner diameter of the insertion device shaft so it may slide under normal manual force applied.

In an exemplary embodiment of the invention, a pusher may be applied to press the piston distally in order to inflate the fixation nail within bone. Optionally, said pusher includes a rod having a diameter equal or lesser than inner diameter of the insertion device shaft. Optionally, the pusher further includes a handle stationed on the rod proximal end in order to allow manual manipulation of the pusher.

In an exemplary embodiment of the invention, a bone access tool is firstly applied in order to pave the way to the metacarpal bone and/or to create a bore and/or widen the medullary canal of the metacarpal prior to nail insertion. Optionally, said bone access tool includes a rod having a sharpened distal tip. In a preferred exemplary embodiment of the invention, the bone access tool may also serve as a pusher for the insertion/inflation device as described above.

The present invention further includes a method of implanting an expandable nail within a small bone, the method comprising:

(a) providing a fixation nail having a longitudinal axis capable of expanding from a first collapsed form to a second preferred expanded form; said nail has an inner bore having at least one inlet; said nail is provided in the first collapsed form; and the nail is provided coupled on its proximal side to an insertion device that includes a longitudinal shaft;

(b) inserting the collapsed fixation nail using the insertion device into an medullar canal of a small bone; and

(c) at least partially expanding the at least part of the fixation nail to a preferred expanded formation and/or until the at least part of the expanded fixation nail is in contact with medullar canal inner surface.

Optionally, said small bone may be but is not limited to a fractured metacarpal, metatarsal, clavicular or phalangeal bone. Optionally, the method includes applying at least part of the devices of the invention formerly described.

Optionally, said fixation bone is expanded by inflation using hydraulic or pneumatic substance. Optionally, said substance is provided by the insertion device. Optionally, the insertion device shaft is hollow and can serve as a channel for pressurizing the fixation nail within bone. Optionally, said hydraulic substance is a fluid containing saline or water. Optionally, the hydraulic fluid is pressurized using a sliding piston under force. Optionally, said force is applied manually using a pusher that includes a rod.

In an exemplary embodiment of the invention, the method further includes a preliminary step that includes accessing the small bone prior to nail insertion. Optionally, this accessing is achieved by using a bone access tool or a stylet that includes a rod having a sharpened distal tip. This preliminary step may include pushing the bone access tool percutaneously into and through the soft tissue surrounding the target small bone until the sharpened tip reaches the bone proximal end. Optionally, this preliminary step may further include pushing said bone access tool into the medullar canal thus creating and/or widening a bore needed in order to insert the collapsed fixation nail.

Optionally, the physician may choose a specific fixation nail from a variety of nails having different designs, materials and/or dimensions. Optionally, the physician may expand the nail to its fully expanded form or alternatively may choose to limit said expanding according to patient and/or small bone and/or fracture condition or form. Optionally, the physician may perform the method while observing using imagery equipment such as an X-ray system.

The present invention further introduces means (i.e. a removal tool) to remove fixation nails that may be added to the physician kit. The fixation nail may be removed either when in at least partial expanded form or be firstly re-collapsed. Optionally, said removal tool includes a rod having connecting means on its distal end capable of connecting with the fixation nail proximal end. Optionally, said connection with nail is achieved by screwing and/or by gripping, but can be maintained by any other connecting means known to art. Optionally, the removal tool distal end further includes a sharpened tip capable of paving the way to the implanted nail proximal end. Optionally, once the removal tool is appropriately connected to the nail proximal end, it can be pulled manually under normal force applied. Optionally, the nail is to be firstly re-collapsed prior to removal. In an alternative exemplary embodiment of the invention, the removal tool is in the form of a slide hammer and further includes an external weight capable of sliding along the rod. Preferably, said weight has a bore having a diameter which is substantially equal or larger than rod's diameter. Optionally, the removal tool further includes a widened proximal end having maximal width larger than weight bore diameter, so weights will be prevented from sliding proximally away from the removal tool rod. Optionally, said widening is a circular nut having an outer diameter larger than weight bore diameter; said nut may be releasably screwed to a threading positioned on rod's proximal end. In a preferred exemplary application, the weight hammers on the rod widened proximal end, so each impact may at least partially shift the fixation nail proximally. Optionally, few impacts are needed to fully remove the nail out of the metacarpal and/or patient body. Optionally, the fixation nail is at least partially expanded when removed.

The present invention further includes a method for manufacturing inflatable fixation nails, the method includes:

(a) providing a plastic deformable tube having a straight axis, a length and a substantially constant inner and outer diameters along its axis; said tube is sealed on its distal end and opened at its proximal end; and

(b) radially pressing at once at least two bar-like forming elements towards the tube center until the tube takes the form of a star or a flower (the number of “petals” is dependant on the number of the pressing elements) while maintaining a maximal diameter which is substantially equal to the original tube outer diameter.

Optionally, the tube is a metal tube, optionally made of stainless steel. Optionally, prior to the radial pressing, the tube is annealed. In an exemplary embodiment of the invention, the method further includes a step in which the “petals” are uniformly pressed in an inward radial direction until the tube reaches a smaller diameter. Preferably, said shorter maximal diameter is the preferred collapsed fixation nail outer diameter.

In a preferred exemplary embodiment of the invention, the plastic deformable tube provided in step (a) above is going through the following procedural steps prior to the radial pressing:

(a1) uniformly narrowing the tube proximal end to smaller inner and outer diameters, while substantially preserving the original tube width; and

(a2) decreasing the width of the tube body while preserving the original width of the tube proximal end.

Optionally, the uniform narrowing of step (a1) is achieved by rotary swaging.

Optionally, the width decrease of step (a2) is achieved by turning and/or uniform circumferential pressing. Optionally, step (a2) further includes chamfering and/or turning the tube distal end to a cone-like shape.

In an exemplary embodiment of the invention, the plastic deformable tube provided in step (a) is manufactured from a single metal piece, including its proximal and/or distal sides. Optionally, no welding and/or soldering are applied throughout the production process of said plastic deformable tube and/or the final product (i.e., the collapsed fixation nail). Alternatively, the plastic deformable tube provided is manufactured from only two metal pieces, when one of said pieces is either the proximal end or the distal end of the tube; said two pieces are welded and/or soldered together to one piece prior to performing step (a) above.

In an exemplary embodiment of the invention, the final width of the nail body is set according to the specific applicability of the nail and the stress magnitude said nail may feel when expanded within bone (higher torsional and/or axial stresses may encourage the use of wider nail bodies.

In one embodiment of the invention, an inflatable nail is used for internally fixating a fractured medullar bone provided in a collapsed formation, the nail is produced by radially crimping a tubular body, said nail when is at least partially inflated, includes longitudinal wrinkles that improve rotational stability and/or friction of the nail within bone.

In an exemplary embodiment of the invention, the nail has a width and excludes longitudinal fastener bars, wherein said width provides enough strength to withstand deformable stresses applied when nail is implanted in an inflated form.

In an exemplary embodiment of the invention, an inflatable nail is used for internally fixating a fractured medullar bone; said nail is capable of expanding to a preferred form by manually operated syringe.

Optionally, said nail is provided attached to said syringe and wherein hydraulic fluid is located in a single volume shared by both nail and syringe.

Optionally, said syringe includes an inner floating slidable piston, that is accessible to a pusher rod which can be manually operated.

Optionally, said nail is expanded from a first collapsed form to a preferred expanded from by applying a certain magnitude of force to the piston by the pusher rod. Optionally, said rod has a sharp distal tip. Optionally, said rod is a stylet capable of being utilized as a bone access tool.

Optionally, the nail has a longitudinal body which is at least partially constructed from metal. Optionally, said at least partially metal nail body is at least partially cold worked.

Optionally, the nail is in collapsed formation. Optionally, said collapsed formation is achieved by radial crimping of a tubular element having an axis a by at least two longitudinal bars that are parallel to said axis. Optionally, said at least two longitudinal bars are evenly spaced along the tubular element circumference. Optionally, said collapsed formation is in the shape of a flower having at least two substantially identical petals evenly spaced along a substantially circular core.

Optionally, the nail, when expanded from said collapsed formation, cannot regain an identical shape to its original tubular shape prior to crimping. Optionally, said expanded nail contains residual wrinkles. Optionally, said wrinkles improve frictional stability with respect to bone medullar canal inner surface.

Optionally, the nail can be expanded non-uniformly along its axis. Optionally, at least one segment of said nail takes the form of its adjacent surrounding medullar canal segment.

Optionally, the nail when in expanded form increases resistance to relative motion among bone portions. Optionally, said relative motion is rotational and/or axial.

Optionally, said bone is a relatively small bone. Optionally, said small bone is a metacarpal bone, or a metatarsal bone, or a phalanx bone, or a clavicular bone.

Optionally, the nail when in collapsed form has maximal width which is lower that 5 mm, optionally lower than 3 mm. Optionally, the nail has total length that is lower than 150 mm, optionally lower than 100 mm, optionally is between 30 to 70 mm.

In one embodiment of the invention, there is provided a method for fixating a fractured bone having a medullar canal, the method comprising:
a. providing a collapsed inflatable nail having an axis, a distal end and a proximal end; said nail is attached on its proximal end to a syringe;
b. inserting the collapsed nail while attached to the syringe into the medullar canal of a the bone by distally pressing the syringe; and
c. inflating at least part of the fixation nail to a preferred expanded formation by manually activating the syringe.

Optionally, said syringe includes an inner floating slidable piston, that is accessible to a pusher rod which can be manually operated.

Optionally, the method further include a preliminary step in which the pusher rod is operated percutaneously to access the bone.

### BRIEF DESCRIPTION OF THE FIGURES

Exemplary non-limiting embodiments of the invention will be described with reference to the following description of embodiments in conjunction with the figures. Identical structures, elements or parts which appear in more than one figure are generally labeled with a same or similar number in all the figures in which they appear, in which:

FIGS. 1A-1D are schematic illustrations of an exemplary expandable nail, in accordance with an exemplary embodiment of the invention;

FIGS. 2A-2B are schematic illustrations of an exemplary expandable nail stationed within an exemplary bone, in accordance with an exemplary embodiment of the invention;

FIGS. 3A-3B illustrate an exemplary expandable nail with an exemplary insertion device, in accordance with an exemplary embodiment of the invention;

FIG. 4 illustrates an expandable bone access tool, in accordance with an exemplary embodiment of the invention;

FIGS. 5A-5B present side view cross sections of exemplary devices presented in FIGS. 2A-2B, in accordance with an exemplary embodiment of the invention;

FIGS. 6A-6B illustrate an exemplary nail extraction device, in accordance with an exemplary embodiment of the invention;

FIGS. 7A-7C illustrate an exemplary preliminary process of an inflatable fixation nail, in accordance with an exemplary embodiment of the invention; and

FIGS. 8A-8C illustrate an exemplary process of an inflatable fixation nail, in accordance with an exemplary embodiment of the invention.

### DETAILED DESCRIPTION OF EXEMPLARY EMBODIMENTS

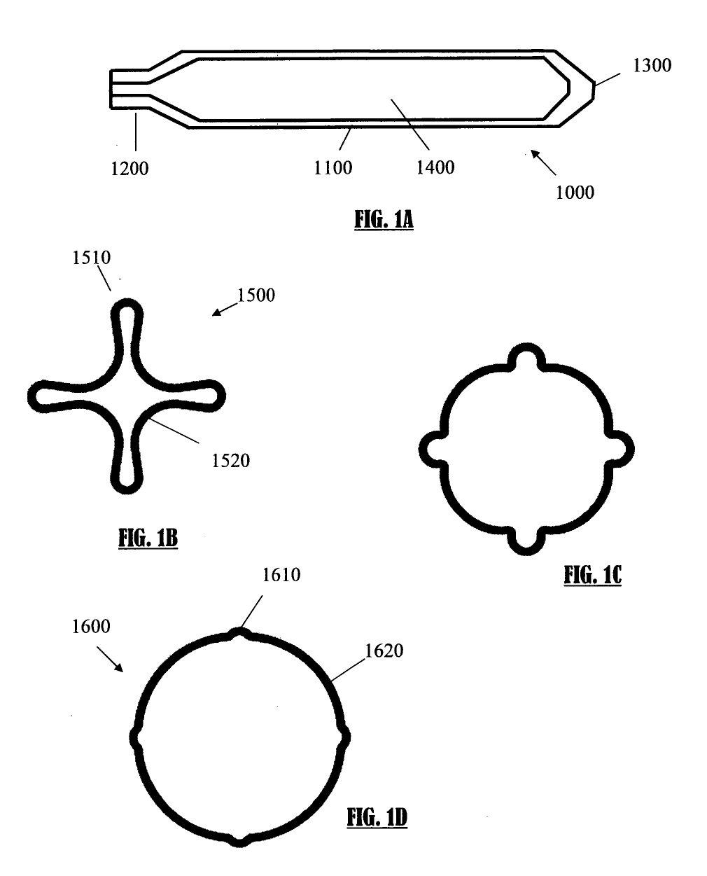

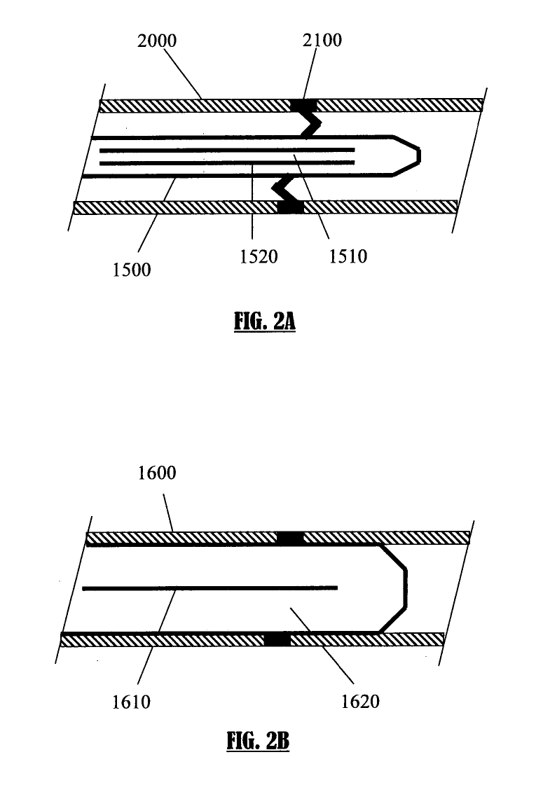

FIGS. 1A-1D schematically illustrate an expandable nail in accordance with an exemplary embodiment of the invention. FIG. 1A presents exemplary fixation nail 1000 in a maximal expanded formation. Nail 1000 includes hollow body 1100 that contains volume 1400, tip 1300 and inlet 1200. In an exemplary embodiment of the invention, body 1100 has a maximal outer diameter in the range of 2-10 mm, optionally 2.5 mm to 7 mm, optionally about 4.5 mm or optionally about 7 mm. Optional length of nail 1000 is 5 mm, optionally about 30 mm, optionally about 70 mm, optionally about 100 mm, optionally about 150 mm or lesser or higher or intermediate values.

In an exemplary embodiment of the invention, a nail having a length of 30-70 mm and maximal diameter of 4.5 mm in fully expanded form is especially intended for treating metacarpal or metatarsal fractures. In an exemplary embodiment of the invention, a nail having a length of about 100 mm and maximal diameter of about 6.8 mm in fully expanded form is especially intended for treating metacarpal or metatarsal fractures.

FIG. 1B illustrates a cross section of nail 1000 in a first collapsed form 1500, whereas same nail in a partial expanded and fully expanded forms are illustrated in FIGS. 1C-1D (respectively). Nail 1000 as presented in FIG. 1B is collapsed to a flower-like shape having four “petals”, although any other number of petals higher than one may be applied. Optionally, collapsed nail 1500 has small outward radii 1510 and large inward radii 1520. Optionally, both radii have been plastically deformed. Preferably, small radii 1510 has higher resistance to bending than larger radii 1520, so when the nail expands to shape 1600, residual fins 1610 remains where radii 1510 were positioned, whereas relatively smooth pattern 1620 between each two adjacent fins 1610 emerges after expansion of larger radii 1520.

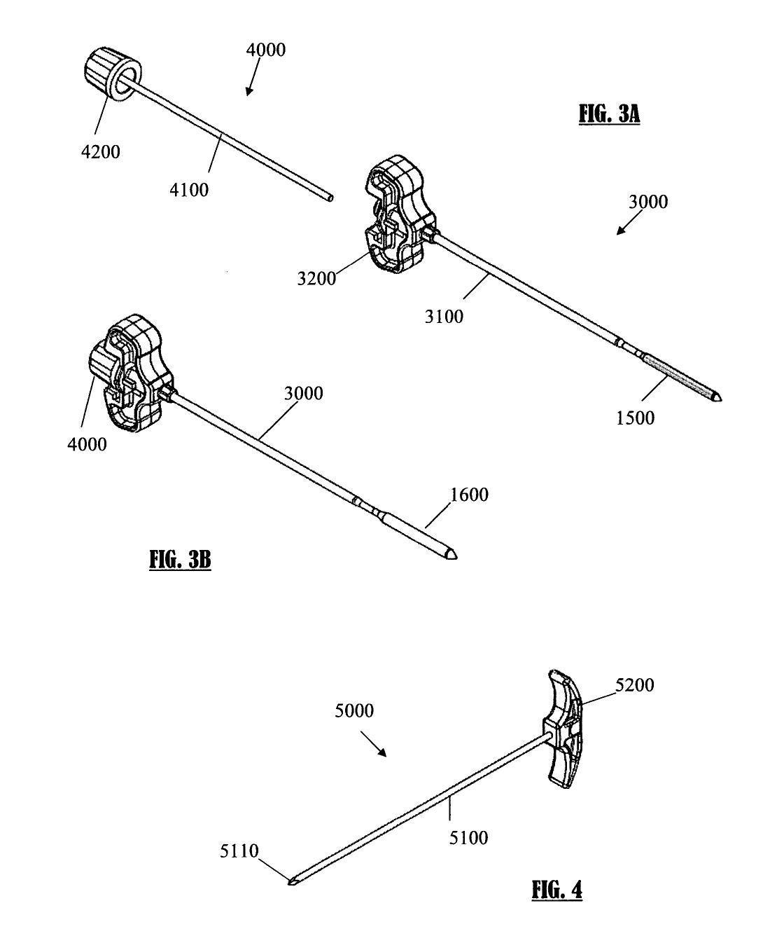

FIGS. 2A-2B illustrate similar nail when in collapsed form 1500 and expanded form 1600 within medullary bone 2000 having fracture line 2100. Preferably, nail 1000 is to be stationed along the two sides of fracture line 2100 in order to holed both sides of bone 2000 and maintain resistance to relative movements. In a preferred exemplary embodiment of the invention, fins 1610 contribute to friction of the nail when stationed within medullar canal of a bone.

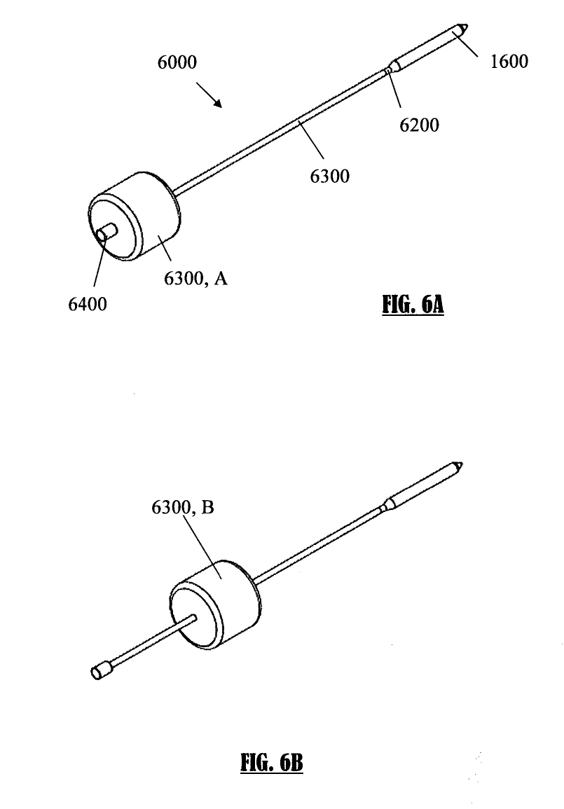

FIGS. 3-5 illustrate exemplary instrumentation kit for inserting and expanding nail 1000 within bone. FIGS. 3A and 5A respectively show isometric view and cut side view of collapsed nail 1500 proximally coupled to insertion device 3000. FIGS. 3B and 5B respectively show isometric view and cut side view of a fully expanded nail 1600 proximally coupled to insertion device 3000. Optionally, the nail is bolted to device 3000 distal end. Optionally, insertion device 3000 includes shaft 3100 and handle 3200. Optionally, shaft 3100 is a hollow tube. In an exemplary embodiment of the invention, device 3000 is also an inflation device capable of inflating nail 1000 from collapsed form 1500 to an expanded form such as fully expanded form 1600. Optionally, device 3000 further include piston 3300. Optionally, device 3000 further contains hydraulic fluid (e.g. saline). In an exemplary embodiment of the invention, the hydraulic fluid occupies at least part of the volume trapped between piston 3300 and nail 1000 distal end.

FIG. 3A also presents exemplary pusher 4000 having rod 4100 and handle 4200. Optionally, rod 4100 has substantially constant outer diameter which is equal or smaller than shaft 3100 inner diameter. In an exemplary embodiment of the invention, pusher 4000 can be used to push piston 3300 distally within shaft 3100 thus reducing the total volume trapped between piston 3300 and nail 1000 distal end. This volume reducing eventually build a higher pressure within said volume until the nail is forced by the hydraulic fluid to expand collapsed nail 1500 to a desired expanded. In an exemplary embodiment of the invention, in order to fully expand the nail, piston 3300 is to travel from a first optional position A (as shown in FIG. 5A) to a second optional position B (as shown in FIG. 5B). Optionally, position A is located at shaft 3100 proximal end, adjacent to handle 3200, whereas position B is located at shaft 3100 distal end, adjacent to coupling point with nail 1000. Optionally, when piston 3300 is in position B, pusher handle 4200 is in contact with insertion device handle 3200, thus optionally preventing any further advancement of piston 3300.

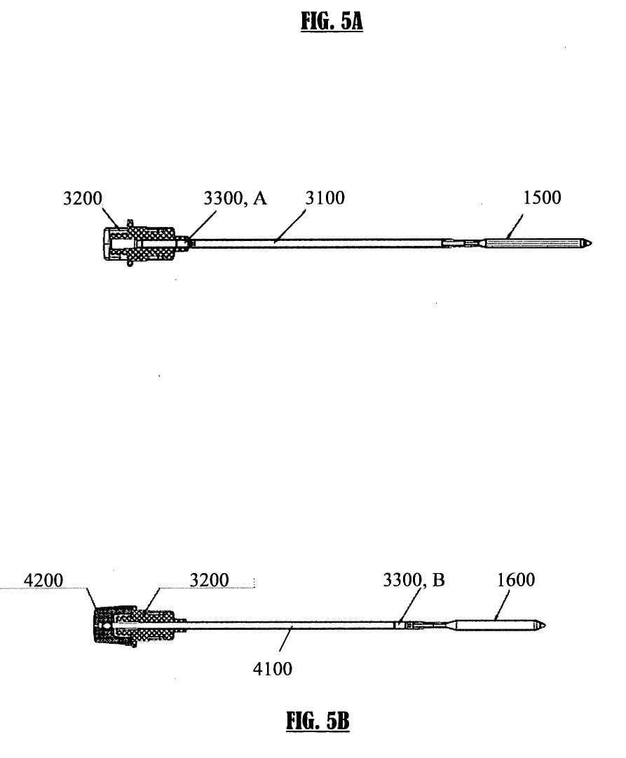

FIG. 4 illustrates exemplary bone access tool 5000 that is capable of creating a percutaneous and/or transdermal path through soft tissue surrounding the target bone, in order to later introduce the nail into bone. Preferably, tool 5000 is designed to be manipulated manually. Optionally, tool 5000 may further create a hole and/or a tunnel within target bone and/or widen its medullar canal prior to nail insertion. Optionally, tool 5000 includes rod 5100 having a sharp tip 5110, and handle 5200. In a preferred exemplary embodiment of the invention, tool 5000 can also serve same functionality as of pusher 4000, i.e. after optional bone accessing step, and after collapsed nail 1500 was properly stationed within bone, the physician can use tool 5000 in order to distally press piston 3300 until nail 1000 is expanded to a preferred form.

FIGS. 6A-6B illustrate exemplary slide hammer device 6000 that can be used for an optional removal of exemplary expanded nail 1600. Nail removal may be desired after bone has been properly healed or in case that another nail should be inserted instead, although the nail can be implanted permanently. Slide hammer 6000 generally includes rod 6100, distal end 6200, weight 6300 and proximal end 6400. Preferably, distal end 6200 includes coupling means to connect with the expanded nail, such as by bolting. Optionally, distal end 6200 further includes a sharp tip that may be applied to re-access the implanted nail percutaneously. Optionally, weight 6400 includes a bore that enables it to slide along rod 6100 under normal manual forces. Optionally, weight 6300 cannot advance proximally after engaging with proximal end 6400. Optionally, distal end 6400 is circular with a diameter larger than weight 6300 bore diameter.

In a preferred exemplary application, by hammering weight 6300 hammers on rod proximal end 6400, the impact may at least partially shift the fixation nail proximally. Optionally, few impacts are needed to fully remove the nail out of the bone and/or patient body.

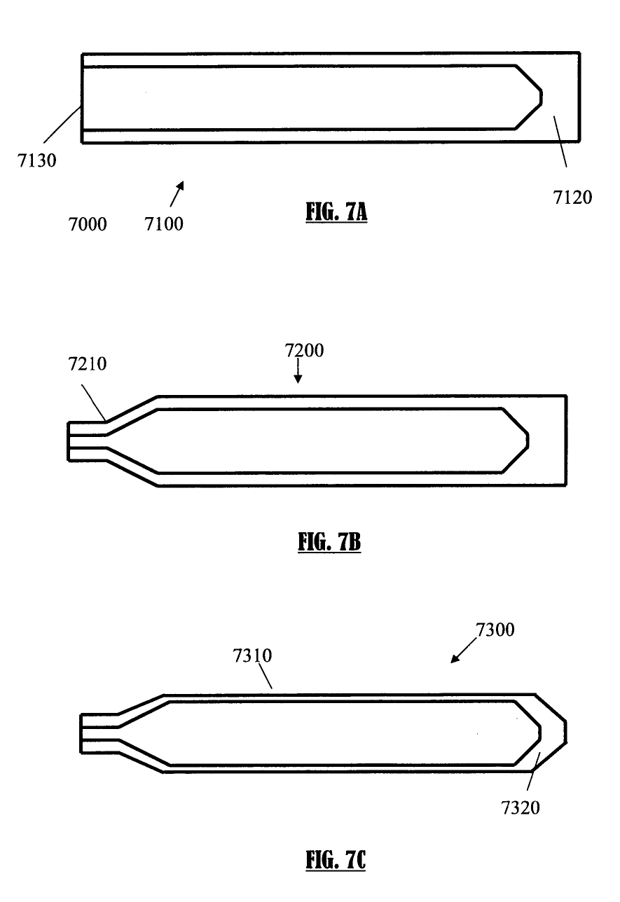

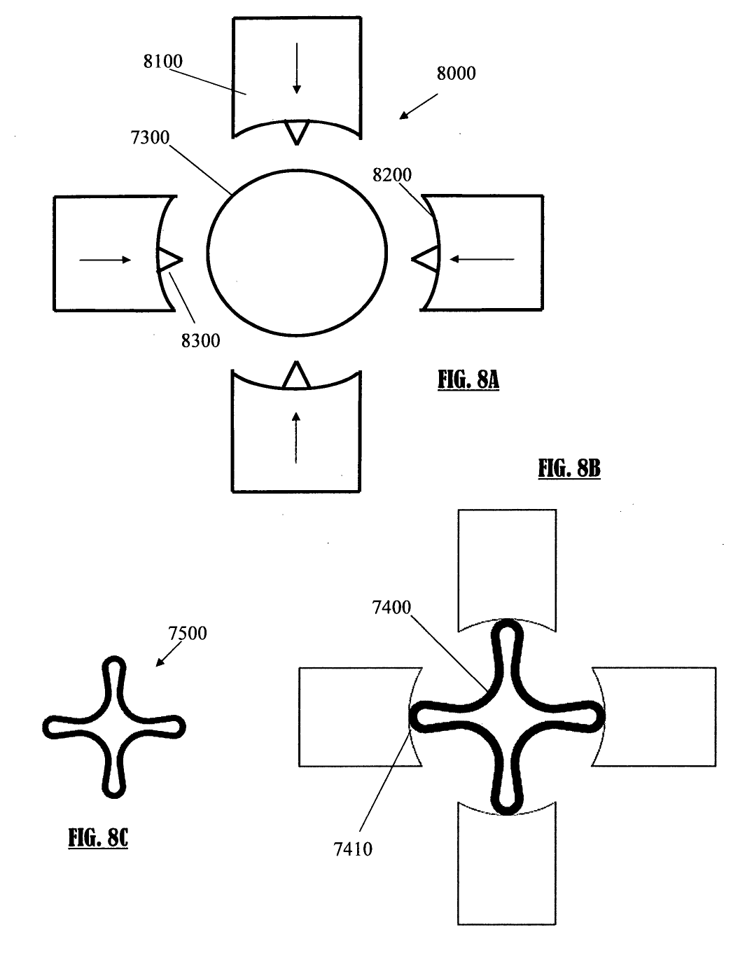

FIGS. 7-8 illustrate an exemplary method of producing exemplary fixation nail 7000, in accordance with the present invention. As illustrated in FIG. 7A, raw material 7100 is provided, which includes tubular body 7110, a closed distal end 7120 and an opened proximal end 7130. In a preferred exemplary embodiment of the invention, raw material 7100 is manufactured from a single piece, for example by partially boring into a tubular rod. Alternatively, raw material 7100 is manufactured by connecting up to two pieces. Optionally, the raw material is metallic. Optionally, the connection of the two pieces is performed by welding and/or soldering.

Optionally, raw material 7100 is then uniformly narrowed at its proximal end 7130, and takes the exemplary general form 7200 as schematically presented in FIG. 7B.

Optionally, form 7200 has a new shaped proximal end 7210 that has smaller inner and outer diameter, while substantially preserving tubular body 7110 original width. Optionally, said narrowing is accomplished by rotary swaging. Optionally, the original width is about 0.1 mm, optionally about 0.3 mm, optionally about 1 mm, optionally about 5 mm, optionally about 10 mm, or lesser or higher or intermediate values. In a preferred exemplary embodiment of the invention, said width is approximately 0.5 mm.

Optionally, form 7200 is then formed to shape 7300 by undergoing a process in which tubular body 7110 width is decreased, while the original width of shaped proximal end 7210 is preserved (as illustrated in FIG. 7C). Optionally, said width decrease is accomplished by turning. Optionally, the new decreased width is about 0.05 to 1 mm, optionally about 0.1 to 0.2 mm. Optionally, distal end 7120 is chamfered and/or lathed to cone-like shape 7320.

Exemplary crimping process of shape 7300 to a collapsed nail formation is illustrated in FIGS. 8A-8C. As illustrated in FIG. 8A, shape 7300 is stationed within crimping device 8000, the device includes plurality of boxes 8100. Optionally, each box 8100 has arc-shaped face 8200 and the boxes are organized so their arc-shaped faces create together a circular shape that substantially resembles to shape 7300 cross-section. Optionally, each arc-shaped face 8200 include one tooth 8300 located on its center. Inward radial forces 8200 are then applied at once on shape 7300 by each one of teeth 8300 until shape 7300 transformed to crimped shape 7400 as illustrated in FIG. 8B. Preferably, shape 7400 includes plurality of protrusions 7410. Teeth 8300 are then pulled out from boxes 8100 and similar force is then applied on shape 7400 from the arc-shaped faces 8200, until crimped shape 7400 is compressed to exemplary collapsed nail shape 7500.

1. An inflatable nail for internally fixating a fractured bone provided in a collapsed formation, wherein said nail includes longitudinal wrinkles when is at least partially inflated.

2. An inflatable nail according to claim 1, wherein said nail is produced by radially crimping a tubular body.

3. An inflatable nail according to claim 1, wherein said bone is a small long bone.

4. An inflatable nail according to claim 1 wherein said bone is a metacarpal/metatarsal bone.

5. An inflatable nail according to claim 1, wherein said nail is provided attached to an insertion device, said insertion device is a syringe, wherein both devices are provided sterile and ready for use.

6. An inflatable nail according to claim 5, wherein hydraulic fluid is located in a single volume shared by both nail and syringe.

7. An inflatable nail according to claim 1, wherein said nail has a longitudinal body which is at least partially constructed from metal.

8. An inflatable nail according to claim 7, wherein said at least partially metal nail body is at least partially cold worked.

9. An inflatable nail according to claim 2, wherein said radial crimping of the tubular body is performed by a machine having at least two longitudinal bars that are parallel to tubular body axis.

10. An inflatable nail according to claim 1, wherein said collapsed formation is in the shape of a flower having at least two substantially identical petals evenly spaced along a substantially circular core.

11. An inflatable nail according to claim 10, wherein said nail, when inflated, cannot regain an identical shape to said tubular body shape.

12. An inflatable nail according to claim 1, wherein said nail can be expanded non-uniformly along its axis.

13. An inflatable nail according to claim 12, wherein at least one segment of said nail takes the form of its adjacent surrounding medullar canal segment.

14. An inflatable nail according to claim 1, wherein said collapsed nail has maximal width which is lower that 5 mm.

15. An inflatable nail according to claim 1, wherein said nail has total length that is lower than 150 mm. An inflatable nail according to claim 1, wherein said nail total length is between 30 to 70 mm.

16. A method for fixating a fractured bone having a medullar canal, the method comprising:
(a) providing a collapsed inflatable nail having an axis, a distal end and a proximal end; said nail is attached on its proximal end to a syringe;
(b) inserting the collapsed nail while attached to the syringe into the medullar canal of a the bone by distally pressing the syringe; and
(c) inflating at least part of the fixation nail to a preferred expanded formation by manually activating the syringe.

17. A method according to claim 16, wherein said syringe includes an inner floating piston, that is accessible to a pusher rod which can be manually operated.

18. A method according to claim 17, wherein the method further include a preliminary step in which the pusher rod is operated percutaneously to access the bone.

19. A method for manufacturing a fixation nail for medullary bones from only a tubular body having distal and proximal ends, wherein said tubular body is produced from a single metal piece.

20. A method according to claim 19, wherein said tubular body is produced without any welding operations.

21. A method for manufacturing a fixation nail for medullary bones from only a tubular body having distal and proximal ends, wherein said tubular body is produced from only two metal pieces welded together.

22. A method according to claim 21, wherein one of said pieces is the tubular body distal end.

23. A method according to claim 21, wherein one of said pieces is the tubular body proximal end.

24. An inflatable fixation nail for medullary bones, the nail has a tubulat wall with a width and excludes longitudinal fastener bars, wherein said width provides enough strength to withstand deformable stresses applied when nail is implanted in an inflated form.
